(19) United States
(12) Patent Application Publication
(10) Pub. No.: US 2020/0129188 A1
(43) Pub. Date: Apr. 30, 2020

(54) DEVICES AND METHODS FOR THE DECORTICATION OF BONE

(71) Applicant: Warsaw Orthopedic, Inc., Warsaw, IN (US)

(72) Inventors: Jeffrey Scifert, Arlington, TN (US); Thomas A. Carls, Memphis, TN (US); Christopher M. Patterson, Olive Branch, MS (US); William A. Rezach, Covington, TN (US); Erick Vasquez, Memphis, TN (US); Kelly Schlachter, Mason, TN (US)

(21) Appl. No.: 16/171,068

(22) Filed: Oct. 25, 2018

(51) Int. Cl. A61B 17/16; A61B 17/32

(52) U.S. Cl. CPC A61B 17/1659; A61B 2017/32007; A61B 17/320068

(57) ABSTRACT

A device for decortication of a bone site is provided. The device comprises an elongated tubular member. The tubular member comprises a rasp configured for oscillating movement of the rasp for decortication of the bone site. The rasp comprises a pressure sensor configured to at least measure pressure from the oscillating movement of the rasp when a predetermined pressure or a predetermined pressure change is measured at the bone site. Methods and kits are also provided.

### BACKGROUND

Bone material and other implantable medical devices for filling and repairing a bone site are often used in orthopedic medicine. While bone wounds can regenerate without the formation of scar tissue, fractures and other orthopedic injuries take a long time to heal, during which time the bone is unable to support physiologic loading without the use of implantable medical devices. Metal pins, screws, rods, plates and meshes are frequently required to replace the mechanical functions of injured bone. However, metal is significantly stiffer than bone. Unlike bone, which can heal small cracks through remodeling to prevent more extensive damage and failure, damaged metal implants can only be replaced or removed. The natural cellular healing and remodeling mechanisms of the body coordinate removal of bone and bone materials by osteoclast cells and formation of bone by osteoblast cells.

Conventionally, bone tissue regeneration is achieved by filling a bone repair site with a bone material (e.g., bone graft). Over time, the bone material is incorporated by the host and new bone remodels the bone material. In order to prepare the bone repair site, a surgeon typically will decorticate the surface of the bone with a device such as a rasp or a drill, increasing the chances of a good outcome of bone graft fusion. Decorticating the bone will cause the bone to bleed which facilitates an increase in osteogenic factors, causing the bone to remodel. However, devices that are used to decorticate bone do not oscillate and also do not dispense bone material.

Therefore, it would be beneficial to provide devices and methods for effectively decorticating and/or dispensing bone material. It would be advantageous if the devices included a rasp and an oscillating feature.

### SUMMARY

Devices and methods for decorticating bone and/or dispensing bone material are provided. In some embodiments, the devices and methods comprise an elongated tubular member. The tubular member comprises a rasp configured for oscillating movement of the rasp for decortication of the bone site. The rasp comprises a pressure sensor configured to at least measure pressure from the oscillating movement of the rasp when a predetermined pressure or a predetermined pressure change is reached at the bone site. In some embodiments, the pressure sensor is coupled to an oscillating tool, the oscillating tool configured to engage the tubular member and reduce or discontinue oscillation of the tubular member when the predetermined pressure or the predetermined pressure change is reached at the bone site.

In some embodiments, a device for decortication of a bone site is provided. The device comprises an elongated tubular member. The tubular member includes a channel configured to receive a wire member having a rasp configured for movement for decortication of the bone site, and the rasp comprises a pressure sensor configured to at least measure pressure from the movement of the wire member when a predetermined pressure or a predetermined pressure change is reached at the bone site.

In some embodiments, a method for decorticating a bone site is provided. The method comprises inserting at the bone site a device for decorticating bone at the bone site, the device comprising an elongated tubular member, the tubular member comprising a rasp configured for oscillating movement of the rasp for decortication of the bone site, and the rasp comprising a pressure sensor configured to at least measure pressure from the oscillating movement of the rasp when a predetermined pressure or a predetermined pressure change is measured at the bone site.

While multiple embodiments are disclosed, still other embodiments of the present disclosure will become apparent to those skilled in the art from the following detailed description. As will be apparent, the disclosure is capable of modifications in various obvious aspects, all without departing from the spirit and scope of the present disclosure. Accordingly, the detailed description is to be regarded as illustrative in nature and not restrictive.

### BRIEF DESCRIPTION OF THE FIGURES

In part, other aspects, features, benefits and advantages of the embodiments will be apparent with regard to the following description, appended claims and accompanying figures.

FIG. 1 is a perspective view of a device for decortication of a bone site. The device includes an elongated tubular member comprising a rasp that oscillates to decorticate a bone site. The rasp includes a pressure sensor that measures pressure from the oscillating movement of the rasp when a predetermined pressure or pressure change is measured at the bone site.

FIG. 2 is a perspective view of the device of FIG. 1 and a processor (e.g., personal computer). The pressure sensor can communicate wirelessly with the processor by a transmitter and wireless connection such as, for example, Bluetooth®.

FIG. 3 is a perspective view of the device of FIG. 1 and a processor (e.g., personal computer). The device can be connected to the personal computer by a wire connection. The wire connection allows the pressure sensor to communicate with the processor.

FIG. 4 is a perspective and partially phantom view of an embodiment of the device which has a flexible portion that functions as a flexible drill bit. The device is shown connected to an oscillating tool such as a drill for oscillating the device. The bone material can be seen pre-loaded into a channel of the tubular member.

FIG. 5 is a perspective view of the device of FIG. 1 disposed with a suction tool to remove decorticated bone particulate from the bone site after the rasp has decorticated the bone site. A vacuum hose and a vacuum source are connected to a proximal end of the suction tool.

FIG. 6 is a perspective and partially phantom view of the device of FIG. 1. In FIG. 6, a flexible plunger is shown disposed within the channel of the tubular member. The flexible plunger is moved in a distal direction to dispense the bone material out of the tubular member and into a surgical site (e.g., vertebrae).

FIG. 7 is a perspective view of the device shown in FIG. 1 having a rasp configuration located on the distal end of the tubular member.

FIG. 8 is a perspective view of the device of FIG. 1 having a rasp configuration located circumferentially about the entire distal end of the elongated tubular member.

FIG. 9 is a front view of a device for decortication of a bone site. The device is shown attached to a power source and an oscillating tool. The oscillating tool includes an ultrasonic energy source and a transducer to supply the device with ultrasonic energy. The device includes an elongated tubular member comprising a rasp for decorticating bone. The rasp includes a pressure sensor that measures pressure from the oscillating movement of the rasp when a predetermined pressure or pressure change is measured at the bone site. The device further comprises a suction tool configured to engage with a proximal end of the elongated tubular member to remove decorticated bone particles from the bone site.

FIG. 10 is a bottom view of the rasp of the device of FIG. 9. In FIG. 10, an end of the suction tool is shown.

FIG. 11 is a perspective view of a device for decorticating bone. The device includes an elongated tubular member having a flexible portion to allow angling of a rasp. The tubular member is connected to an oscillating tool such as a drill to supply the device with oscillation for decorticating the bone.

FIG. 12 is a perspective view of a device for decorticating bone. The device includes an elongated tubular member having a flexible portion to allow angling of a rasp. The device includes a sleeve that is configured to retract to expose at least a portion of the rasp. The tubular member is connected to an oscillating tool such as a drill to supply the device with oscillation for decorticating the bone. In FIG. 12, the sleeve is shown covering the entire rasp.

FIG. 13 is a perspective view of the device of FIG. 12. In FIG. 13, the device is shown connected to an oscillating tool such as a drill and the sleeve is shown retracted such that at least a portion of the rasp can be used to decorticate the bone.

FIG. 14 is a perspective view of a device for decorticating bone, at least one guidewire having a loop and the bone site. The device includes an elongated tubular member comprising a rasp and a channel. The at least one guidewire is configured to guide the placement of the device into or near a bone site such that bone can be decorticated and bone material can be dispensed from the device.

FIG. 15 is a perspective view of the device of FIG. 14, the at least one guidewire having a loop and the bone site. In this FIG., the bone material is dispensed into the bone site.

FIG. 16 is a perspective view of an embodiment of a device for decorticating bone. The device comprises an elongated tubular member having a channel configured to receive a wire member having a rasp. The rasp is configured for movement for decortication of the bone site, and the rasp comprises a pressure sensor configured to at least measure pressure from the movement of the wire member when a predetermined pressure or a predetermined pressure change is measured at the bone site. In FIG. 16, the bone site is the transverse process.

FIG. 17 is a block diagram of a method of decorticating a bone site and then dispensing bone material to the bone site by implementing at device and at least one guidewire.

FIG. 18 is a block diagram of a method of decorticating bone by implementing a device comprising a tubular member and an oscillating rasp.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

### Definitions

It is noted that, as used in this specification and the appended claims, the singular forms “a,” “an,” and “the,” include plural referents unless expressly and unequivocally limited to one referent.

The term “allograft” refers to a graft of tissue obtained from a donor of the same species as, but with a different genetic make-up from, the recipient, as a tissue transplant between two humans.

The term “autologous” refers to being derived or transferred from the same individual's body, such as for example an autologous bone marrow transplant.

The term “xenograft” refers to tissue or organs from an individual of one species transplanted into or grafted onto an organism of another species, genus, or family.

The term “mammal” refers to organisms from the taxonomy class “mammalian,” including, but not limited to, humans; other primates, such as chimpanzees, apes, orangutans and monkeys; rats, mice, cats, dogs, cows, horses, etc.

The term “patient” refers to a biological system to which a treatment can be administered. A biological system can include, for example, an individual cell, a set of cells (e.g., a cell culture), an organ, or a tissue. Additionally, the term “patient” can refer to animals, including, without limitation, humans.

The term “bone material” includes natural and/or inorganic material such as, for example, inorganic ceramic and/or bone substitute material. The bone material can also include natural bone material such as, for example, bone which is cortical, cancellous or cortico-cancellous of autogenous, allogenic, xenogenic, or transgenic origin. In some embodiments, bone material can include demineralized bone material such as, for example, substantially demineralized bone material, partially demineralized bone material, or fully demineralized bone material.

“Demineralized” as used herein, refers to any material generated by removing mineral material from tissue, e.g., bone tissue. In certain embodiments, the demineralized compositions described herein include preparations containing less than 5% calcium and preferably less than 1% calcium by weight. Partially demineralized bone (e.g., preparations with greater than 5% calcium by weight but containing less than 100% of the original starting amount of calcium) is also considered within the scope of the application. In some embodiments, demineralized bone has less than 95% of its original mineral content.

In some embodiments, demineralized bone has less than 95% of its original mineral content. In some embodiments, demineralized bone has less than 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6 and/or 5% of its original content. In some embodiments, “demineralized” is intended to encompass such expressions as “substantially demineralized,” “superficially demineralized,” “partially demineralized,” “surface demineralized,” and “fully demineralized.”

“Partially demineralized” is intended to encompass “surface demineralized.” “Partially demineralized bone” is intended to refer to preparations with greater than 5% calcium by weight but containing less than 100% of the original starting amount of calcium. In some embodiments, partially demineralized comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 and/or 99% of the original starting amount of calcium.

In some embodiments, the demineralized bone may be surface demineralized from about 1-99%. In some embodiments, the demineralized bone is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 and/or 99% surface demineralized. In various embodiments, the demineralized bone may be surface demineralized from about 15-25%. In some embodiments, the demineralized bone is 15, 16, 17, 18, 19, 20, 21, 22, 23, 24 and/or 25% surface demineralized.

“Superficially demineralized” as used herein, refers to bone-derived elements possessing at least about 90 weight percent of their original inorganic mineral content, the expression “partially demineralized” as used herein refers to bone-derived elements possessing from about 8 to about 90 weight percent of their original inorganic mineral content, and the expression “fully demineralized” as used herein refers to bone containing less than 8% of its original mineral context.

“Demineralized bone matrix” as used herein, refers to any material generated by removing mineral material from bone tissue. In preferred embodiments, the DBM compositions as used herein include preparations containing less than 5% calcium and preferably less than 1% calcium by weight.

“Biocompatible” as used herein, refers to materials that, upon administration in vivo, do not induce undesirable long-term effects.

“Osteoconductive” as used herein, refers to the ability of a non-osteoinductive substance to serve as a suitable template or substance along which bone may grow.

“Osteogenic”, as used herein, refers to the ability of an agent, material, or implant to enhance or accelerate the growth of new bone tissue by one or more mechanisms such as osteogenesis, osteoconduction, and/or osteoinduction.

“Osteoinductive” as used herein, refers to the quality of being able to recruit cells from the host that have the potential to stimulate new bone formation. Any material that can induce the formation of ectopic bone in the soft tissue of an animal is considered osteoinductive. For example, most osteoinductive materials induce bone formation in athymic rats when assayed according to the method of Edwards et al., “Osteoinduction of Human Demineralized Bone: Characterization in a Rat Model,” Clinical Orthopaedics & Rel. Res., 357:219-228, December 1998, incorporated herein by reference.

The terms “upper”, “lower”, “top”, “bottom”, “side”, “proximal”, “distal” and so forth have been used herein merely for convenience to describe the present invention and its parts as oriented in the drawings. It is to be understood, however, that these terms are in no way limiting to the disclosure since the decorticating devices described herein may obviously be disposed in different orientations when in use.

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding the numerical ranges and parameters set forth herein, the broad scope of the invention is an approximation; the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Reference will now be made in detail to certain embodiments of the disclosure, examples of which are illustrated in the accompanying figures. While the disclosure will be described in conjunction with the illustrated embodiments, it will be understood that they are not intended to limit the disclosure to those embodiments. On the contrary, the disclosure is intended to cover all alternatives, modifications, and equivalents that may be included within the disclosure as defined by the appended claims.

The headings below are not meant to limit the disclosure in any way; embodiments under any one heading may be used in conjunction with embodiments under any other heading.

### Decorticating Devices

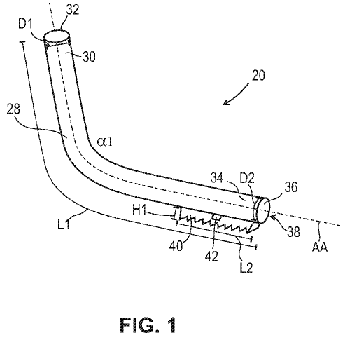

A device 20, as shown in FIGS. 1-8, is provided for decorticating bone 22 at a bone site 24. The device is configured to prepare a bone by decorticating (e.g., roughening) the surface of the bone to denuter the bone. In some embodiments, decortication will remove portions of the bone surface. In some embodiments, denutering the bone will increase osteogenic factors to cause bone to remodel. In some embodiments, the device decorticates bone and also allows for dispensing bone material 26, such as bone graft to the bone site. In some embodiments, the device can be used in minimally invasive surgery (MIS) procedures such as MIS posterolateral procedures. In some embodiments, sites of the surgery can include, but are not limited to, injury or defects brought about during the course of surgery, infection, malignancy or developmental malformation. In some embodiments, bones which can be repaired with the device and bone material can include, but are not limited to the ethmoid; frontal; nasal; occipital; parietal; temporal; mandible; maxilla; zygomatic; cervical vertebra; thoracic vertebra; lumbar vertebra; sacrum; rib; sternum; clavicle; scapula; humerus; radius; ulna; carpal bones; metacarpal bones; phalanges; ilium; ischium; pubis; femur tibia; fibula; patella; calcaneus; tarsal and metatarsal bones.

The device includes an elongated tubular member 28. In some embodiments, the tubular member is flexible. In some embodiments, the tubular member can be a cannula. The tubular member comprises a proximal end 30 defining a proximal opening 32, a distal end 34 defining a distal opening 36, a channel 38 and a longitudinal axis AA disposed therebetween. The proximal opening is configured to receive bone material, and the distal opening is configured to dispense the bone material. The proximal opening has a diameter D1 and the distal opening has a diameter D2, as shown in FIG. 1. In some embodiments, diameters D1 and D2 can be the same size, D1 can be greater than D2 or D2 can be greater than D1. In some embodiments, D1 and D2 can be from about 4 mm to about 30 mm, from about 4 mm to about 20 mm, from about 4 mm to about 10 mm, from about 10 mm to about 30 mm, from about 10 mm to about 20 mm, from about 10 to about 15 mm, from about 15 mm to about 30 mm, from about 15 to about 25 mm, from about 15 mm to about 20 mm, from about 20 mm to about 30 mm or from about 20 mm to about 25 mm. In some embodiments, diameters D1 and D2 can be from about 4, 5, 6, 7, 8, 9, 10, 11, 12, 13, 14, 15, 16, 17, 18, 19, 20, 21, 22, 23, 24, 25, 26, 27, 28, 29 to about 30 mm.

In some embodiments, the tubular member can have a length L1, as shown in FIG. 1. The length L1 can be from about 1 inch to about 20 inches, from about 1 to about 15 inches, from about 1 to about 10 inches, from about 1 to about 5 inches, from about 5 to about 20 inches, from about 5 to about 15 inches, from about 5 to about 10 inches, from about 10 to about 20 inches, from about 10 to about 15 inches, or from about 15 to about 20 inches. In some embodiments, the length L1 can be from about 1, 2, 3, 4, 5, 6, 7, 8, 9, 10, 11, 12, 13, 14, 15, 16, 17, 18, 19 to about 20 inches.

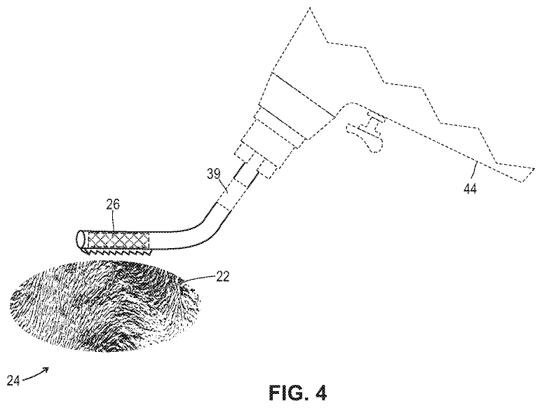

In some embodiments, the tubular member can be flexible having a flexible portion 39 and/or can be angled. The flexible portion, as shown in FIG. 4, functions as a flexible drill bit to allow angling of a rasp 40 to enhance the rasping of bone by the rasp. In this way, the device can be positioned at or adjacent to a confined area of the bone (e.g., the spine). In some embodiments, the flexible portion can be any particular length, and can be manufactured at a particular length depending on the location of the bone site. In some embodiments, the flexible portion can be made from the same or different material as the remainder of the tubular member.

In some embodiments, the tubular member can be straightened even when the tubular member is angled in a resting configuration. In some embodiments, the tubular member can be angled and have an angle α1 from about 10 to about 60 degrees. In some embodiments, α1 can be from about 10, 12, 14, 16, 18, 20, 22, 24, 26, 28, 30, 32, 34, 36, 38, 40, 42, 44, 46, 48, 50, 52, 54, 56, 58 to about 60 degrees.

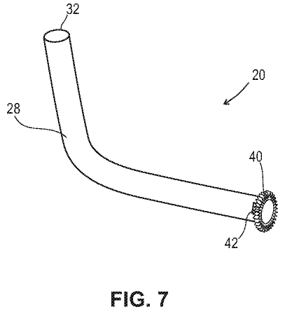

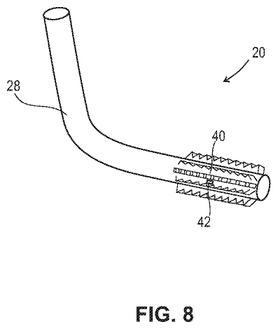

The tubular member comprises rasp 40 that is configured for oscillating movement for decortication of the bone site, as shown in FIGS. 1-8. In some embodiments, the rasp is located on an exterior surface of the distal end of the tubular member. In some embodiments, the rasp is located on an exterior surface of the distal end of the tubular member and runs parallel with the distal end and perpendicular to the distal opening of the tubular member, as shown in FIG. 1. In some embodiments, the rasp is located on an exterior surface of the distal end of the tubular member and is disposed circumferentially about the distal opening of the tubular member, as shown in FIG. 7. In some embodiments, the rasp is located on an exterior surface of the entire distal end of the tubular member and is disposed circumferentially about the distal end, as shown in FIG. 8.

In some embodiments, the rasp can have a length L2, as shown in FIG. 1. In some embodiments, the length L2 can be from about 2 mm to about 50 mm. In some embodiments, the length L2 can be from about 2 mm to about 40 mm, from about 2 mm to about 30 mm, from about 2 mm to about 20 mm, from about 2 mm to about 10 mm, from about 10 mm to about 50 mm, from about 10 mm to about 40 mm, from about 10 mm to about 30 mm, from about 10 mm to about 20 mm, from about 20 mm to about 50 mm, or from about 20 mm to about 40 mm. In some embodiments, the length L2 can be from about 2, 4, 6, 8, 10, 12, 14, 16, 18, 20, 22, 24, 26, 28, 30, 32, 34, 36, 38, 40, 42, 44, 46, 48 to about 50 mm.

In some embodiments, the rasp can have a certain height H1, as shown in FIG. 1. In some embodiments, the height H1 can be from about 1 mm to about 8 mm, from about 1 mm to about 6 mm, or from about 1 mm to about 4 mm. In some embodiments, the height H1 can be from about 1, 2, 3, 4, 5, 6, 7 to about 8 mm.

In some embodiments, the rasp has various surface configurations and can include, but is not limited to, a grid, a mesh configuration, a honeycomb weave, rasp studs, raised protrusions, raised projections, hooks, straight blades, forward cutting blades, backward-cutting blades, and transverse cutting blades, angled teeth, flat teeth, curved teeth, beveled teeth, gullets, a scouring surface having a selected grit (e.g., the scouring surface is similar to sand paper texture), ridges, grooves, or a combination thereof. In some embodiments, the rasp can include a plurality of projections and the projections can be the same of different sizes. In some embodiments, the bone decorticated or scraped off during the use of the device can be contained within the grid, mesh configuration, honeycomb weave, gullets, and/or grooves of the rasp to reduce a risk of emboli or thromboses in the patient. In some embodiments, the teeth and/or the blades can be angled in the same or different directions. In some embodiments, the rasp can be made of one or more materials, such as, for example, metals such as titanium and/or steel.

In some embodiments, the rasp can be monolithic with the distal end of the tubular member. For example, the rasp can be formed from the tubular member and can be made from the same material. In some embodiments, the rasp and the tubular member can be made via over-molding techniques or 3-D printing such that the rasp and the tubular member are formed from either the same or different material.

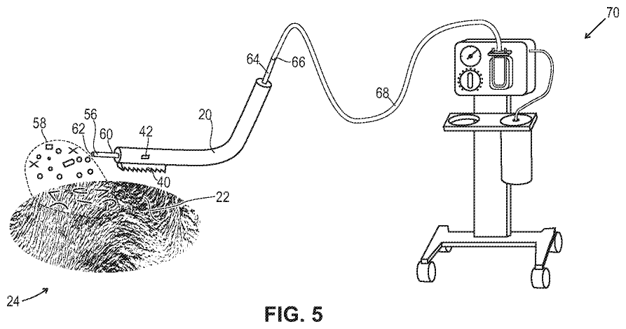

The rasp comprises a pressure sensor 42 configured to at least measure pressure from the oscillating movement of the rasp when a predetermined pressure or a predetermined pressure change is measured at the bone site. In some embodiments, the pressure sensor can be disposed on or adjacent to the rasp. In some embodiments, the rasp and/or other portions of the device can include one or more pressure sensors, as shown in FIG. 5. In some embodiments, the sensor can be square, round, circular or rectangular in shape. In some embodiments, the sensor can be embedded into a groove or indent in a surface of the rasp or the distal end of the tubular member such that the sensor does not get damaged when the rasp is in use. In some embodiments, the sensor is made to be waterproof via a polymer coating or film to prevent damage of the sensor during device use.

In some embodiments, the rasp and/or the device can include 1, 2, 3, 4, 5, 6, 7, 8, 9 or 10 sensors. In some embodiments, the rasp and/or the device can include additional sensors such as, for example, an image sensor, a motion sensor, a temperature sensor, and/or a humidity sensor. In some embodiments, the device can also be configured with a navigational system.

In some embodiments, the pressure sensor is coupled to an oscillating tool, such as a surgical drill 44. In some embodiments, the oscillating tool can be an ultrasound probe, as described herein. In some embodiments, the oscillating toll can be a ratchet or other rotatable tool to facilitate rotation of the device for decortication of the bone site. In some embodiments, the rasp rotates about longitudinal axis AA. The oscillating tool is configured to engage the proximal end of the tubular member and the flexible portion enables the rasp to rotate during oscillation. The oscillating tool will reduce or discontinue oscillation of the tubular member/rasp when the predetermined pressure or the predetermined pressure change is measured at the bone site by the sensor. In some embodiments, the device is oscillated until a predetermined pressure is reached, such as, for example, when the pressure is at a maximum in cortical bone. Once the rasp passes through the cortical bone, the pressure will drop, and the rasp will discontinue oscillation. In some embodiments, pressure change detected by the pressure sensor indicates the type of bone that the device is encountering.

In some embodiments, the rasp is configured to oscillate axially relative to longitudinal axis AA of the tubular member, radially relative to longitudinal axis AA of the tubular member, and/or diagonally relative to longitudinal axis AA of the tubular member. In some embodiments, the rasp is configured to vibrate causing the rasp to move in multiple directions.

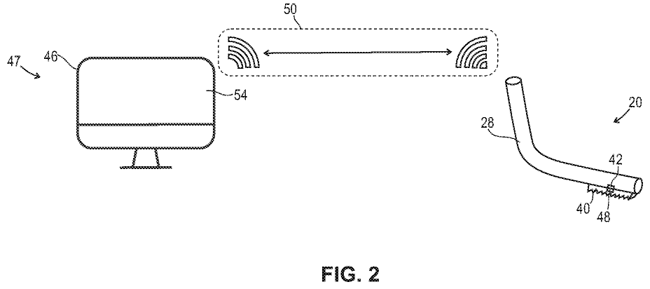

In some embodiments, the pressure sensor is configured to transmit electronic signals to a processor 46 of a computer 47. The pressure sensor can communicate wirelessly with the processor by a transmitter 48 and a wireless connection 50 such as, for example, Bluetooth®, as shown in FIG. 2. In some embodiments, the transmitter is located within the sensor. The Bluetooth® attempts to establish a wireless connection with the processor which serves as the Bluetooth® receiver and when a connection occurs, pressure sensor data will be streamed to the processor.

In some embodiments, the computer can be one of a plurality of devices such as, for example, network/stand-alone computers, personal digital assistants (PDAs), WebTV (or other Internet-only) terminals, set-top boxes, cellular/phones, screen phones, pagers, blackberry, smart phones, iPhone, iPad, table, peer/non-peer technologies, kiosks, or other known (wired or wireless) communication devices, etc.

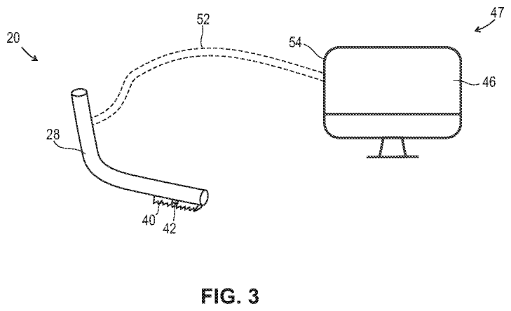

In some embodiments, the device alternatively can be connected to the computer by a wire connection 52, as shown in FIG. 3. The wire connection allows the pressure sensor to communicate with the computer.

In some embodiments, the computer can be loaded with a software program such that data collected from the pressure sensor or other sensors can be stored and interfaces with the practitioner such that data can be searched, retrieved and displayed by the practitioner. In some embodiments, the data may be downloaded in one or more textual/graphical formats (e.g., RTF, PDF, TIFF, JPEG, STL, XML, XDFL, TXT etc.), or set for alternative delivery to the computer. The data may be displayed at a user interface, which allows viewing on the same display, such a monitor 54, as shown in FIGS. 2 and 3.

In some embodiments, the user interface can include one or more display devices (e.g., CRT, LCD, or other known displays) or other output devices (e.g., printer, etc.), and one or more input devices (e.g., keyboard, mouse, stylus, touch screen interface, or other known input mechanisms) for facilitating interaction of the practitioner with the data from the sensor. The user interface may be directly coupled to a database or directly coupled to a network server system via the Internet or cloud computing.

In some embodiments, the user interface may be implemented as a graphical user interface (GUI) containing a display or the like, or may be a link to other user input/output devices known in the art. Individual or of a plurality of devices (e.g., network/stand-alone computers, personal digital assistants (PDAs), WebTV (or other Internet-only) terminals, set-top boxes, cellular/phones, screen phones, pagers, blackberry, smart phones, iPhone, iPad, table, peer/non-peer technologies, kiosks, or other known (wired or wireless) communication devices, etc.) may similarly be used to execute one or more computer programs (e.g., universal Internet browser programs, dedicated interface programs, etc.) to allow the user to monitor oscillation or drilling in the manner described. Database hardware and software can be developed for access by the user through personal computers, mainframes, and other processor-based devices. A user may access the data stored locally on hard drives, CD-ROMs, stored on network storage devices through a local area network, or stored on remote database systems through one or more disparate network paths (e.g., the Internet).

In some embodiments, the device can engage with a suction tool 56 via the distal end opening and the channel of the tubular member to remove decorticated bone particulate 58 from the bone site, as shown in FIG. 5. The suction tool includes a proximal end 60 having an opening 62 and a distal end 64 having an opening 66 that is attached to a vacuum hose 68 and a vacuum source 70.

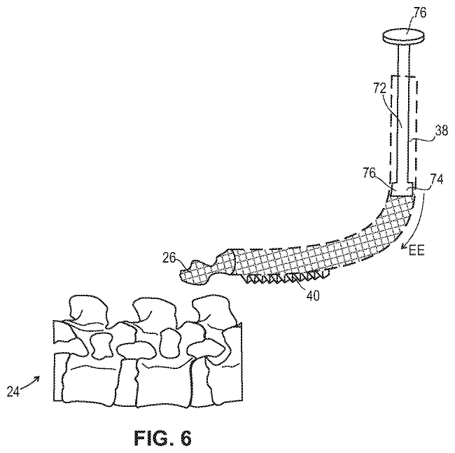

In some embodiments, the device engages with a plunger 72 to dispense the bone material out of the tubular member, as shown in FIG. 6. The plunger includes a proximal end 74 and a distal end 76. In some embodiments, the proximal end includes a tip 76 configured to engage with the bone material. In some embodiments, the tip can have various geometries and sizes that are tailored for varying viscosities of bone material. In some embodiments, the tip of the plunger can be square, rectangular, round, plug, or disc shaped. The plunger length can be smaller, larger or the same size as the tubular member.

The plunger is moved in a downward or distal direction, as shown by arrow EE in FIG. 6 to dispense the bone material loaded within the channel from the distal opening or distal end of the tubular member and into the bone site. In some embodiments, the plunger is flexible and at least a portion of the plunger is configured to slide within the channel of the tubular member.

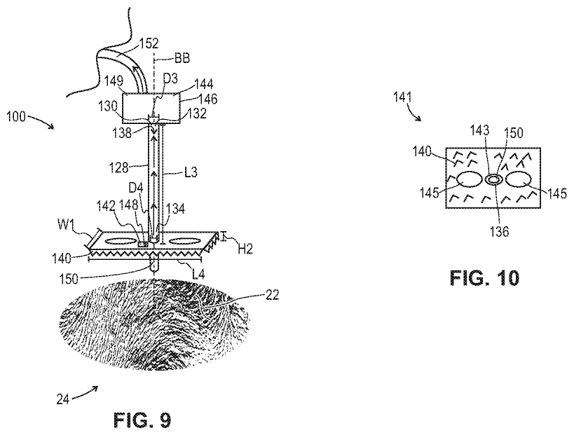

In some embodiments, as shown in FIGS. 9-10, a device 100 for decorticating bone is provided, similar to device 20 of FIGS. 1-8. The device is configured to prepare a bone site by decorticating the surface of the bone to denuter the bone. The device includes an elongated tubular member 128, similar to tubular member 28 of FIGS. 1-8. In some embodiments, the tubular member is flexible. The tubular member comprises a proximal end 130 defining a proximal opening 132, a distal end 134 defining a distal opening 136, a channel 138 and a longitudinal axis BB disposed therebetween. In some embodiments, the proximal opening is configured to receive bone material, and the distal opening is configured to dispense the bone material and to engage a suction tool, as described below.

In some embodiments, the proximal opening has a diameter D3 and the distal opening has a diameter D4, as shown in FIG. 9. In some embodiments, diameters D3 and D4 can be the same size, D3 can be greater than D4 or D4 can be greater than D3. In some embodiments, D3 and D4 can be from about 4 mm to about 30 mm, from about 4 mm to about 20 mm, from about 4 mm to about 10 mm, from about 10 mm to about 30 mm, from about 10 mm to about 20 mm, from about 10 to about 15 mm, from about 15 mm to about 30 mm, from about 15 to about 25 mm, from about 15 mm to about 20 mm, from about 20 mm to about 30 mm or from about 20 mm to about 25 mm. In some embodiments, diameters D3 and D4 can be from about 4, 5, 6, 7, 8, 9, 10, 11, 12, 13, 14, 15, 16, 17, 18, 19, 20, 21, 22, 23, 24, 25, 26, 27, 28, 29 to about 30 mm.

In some embodiments, the tubular member can have a length L3, as shown in FIG. 9. The length L3 can be from about 1 inch to about 20 inches, from about 1 to about 15 inches, from about 1 to about 10 inches, from about 1 to about 5 inches, from about 5 to about 20 inches, from about 5 to about 15 inches, from about 5 to about 10 inches, from about 10 to about 20 inches, from about 10 to about 15 inches, or from about 15 to about 20 inches. In some embodiments, the length L3 can be from about 1, 2, 3, 4, 5, 6, 7, 8, 9, 10, 11, 12, 13, 14, 15, 16, 17, 18, 19 to about 20 inches.

The tubular member comprises a rasp 140, similar to rasp 40 of FIGS. 1-8, and is configured for oscillating movement to decorticate bone at the bone site, as shown in FIG. 9. In some embodiments, the rasp is located on an exterior surface of the distal end of the tubular member. In some embodiments, the rasp is located on an exterior surface of the distal end of the tubular member and runs transverse relative to the distal end of the tubular member, as shown in FIG. 1. In some embodiments, as shown in FIGS. 9 and 10, the rasp can be a part of a plate 141 that engages with the distal end of the tubular member. In some embodiments, the plate containing the rasp can be fixed to the distal end of the tubular member via a snap engagement, a friction fit engagement, a male/female engagement, can engage via an adhesive, can engage via ultrasonic welding, or a combination thereof.

In some embodiments, the plate can be a particular shape and size. In some embodiments, the plate can be square, rectangular, circular, triangular, oval, irregularly shaped, pentagonal, hexagonal, or any other shape, according to the needs of a particular application.

In some embodiments, the plate containing the rasp can have a length L3, as shown in FIG. 9. In some embodiments, the length L2 can be from about 2 mm to about 50 mm. In some embodiments, the length L3 can be from about 2 mm to about 40 mm, from about 2 mm to about 30 mm, from about 2 mm to about 20 mm, from about 2 mm to about 10 mm, from about 10 mm to about 50 mm, from about 10 mm to about 40 mm, from about 10 mm to about 30 mm, from about 10 mm to about 20 mm, from about 20 mm to about 50 mm, or from about 20 mm to about 40 mm. In some embodiments, the length L3 can be from about 2, 4, 6, 8, 10, 12, 14, 16, 18, 20, 22, 24, 26, 28, 30, 32, 34, 36, 38, 40, 42, 44, 46, 48 to about 50 mm.

In some embodiments, the rasp can have a certain height H2, as shown in FIG. 9. In some embodiments, the height H2 can be from about 1 mm to about 8 mm, from about 1 mm to about 6 mm, or from about 1 mm to about 4 mm. In some embodiments, the height H2 can be from about 1, 2, 3, 4, 5, 6, 7 to about 8 mm.

In some embodiments, the plate containing the rasp includes a width W1. In some embodiments, the width W1 can be from about 2 to about 50 mm. In some embodiments, the width W1 can be from about 2 mm to about 40 mm, from about 2 mm to about 30 mm, from about 2 mm to about 20 mm, from about 2 mm to about 10 mm, from about 10 mm to about 50 mm, from about 10 mm to about 40 mm, from about 10 mm to about 30 mm, from about 10 mm to about 20 mm, from about 20 mm to about 50 mm, or from about 20 mm to about 40 mm. In some embodiments, the length W1 can be from about 2, 4, 6, 8, 10, 12, 14, 16, 18, 20, 22, 24, 26, 28, 30, 32, 34, 36, 38, 40, 42, 44, 46, 48 to about 50 mm.

The plate includes a centrally located opening 143 that is configured for engagement with the distal end of the tubular member. In some embodiments, the plate includes one or more additional openings 145.

In some embodiments, the rasp has various surface configurations and materials, and can include the surface configurations and materials disclosed above with regard to rasp 40 of FIGS. 1-8.

In some embodiments, the rasp can be monolithic with the distal end of the tubular member. For example, the rasp can be formed from the tubular member and can be made from the same material. In some embodiments, the rasp and the tubular member can be made via over-molding techniques or 3-D printing such that the rasp and the tubular member are formed from either the same or different material.

The rasp comprises a pressure sensor 142 that includes a transmitter 148. The pressure sensor is configured to at least measure pressure from the oscillating movement of the rasp when a predetermined pressure or a predetermined pressure change is measured at the bone site. The pressure sensor and the transmitter are the same as pressure sensor 42 and transmitter 48 described above with regard to FIGS. 1-8.

In some embodiments, the rasp and/or the device can include 1, 2, 3, 4, 5, 6, 7, 8, 9 or 10 sensors. In some embodiments, the rasp and/or the device can include additional sensors such as, for example, an image sensor, a motion sensor, a temperature sensor, and/or a humidity sensor.

In some embodiments, the pressure sensor is coupled to an oscillating tool 144. In some embodiments, the oscillating tool can be an ultrasound probe. In some embodiments, the oscillating tool comprises an ultrasonic energy source 146 configured to supply the rasp with an ultrasonic energy. The ultrasonic energy source is coupled to a transducer 149 to transfer the ultrasonic energy from the ultrasonic energy source to the rasp. It is to be understood that the oscillating tool can also be the oscillating tools described herein.

In some embodiments, the rasp rotates about longitudinal axis BB. The oscillating tool will reduce or discontinue oscillation of the tubular member when the predetermined pressure or the predetermined pressure change is measured at the bone site by the sensor. In some embodiments, the practitioner applies pressure to the plate having the rasp manually.

In some embodiments, the rasp is configured to oscillate axially relative to longitudinal axis BB of the tubular member, radially relative to longitudinal axis BB of the tubular member, and/or diagonally relative to longitudinal axis BB of the tubular member. In some embodiments, the rasp is configured to vibrate causing the rasp to move in multiple directions.

In some embodiments, the pressure sensor is configured to transmit electronic signals to a processor of a computer, as described above with regard to FIGS. 1-8.

In some embodiments, the device comprises a suction tool 150, similar to suction tool 56 of FIG. 5, that is configured to engage the distal end of the tubular member and the channel of the tubular member to remove decorticated bone particles from the bone site. The suction tool can be moved out of the opening 143 of the plate such that decorticated particles can be removed from the bone site. The suction tool can be attached to a vacuum source 152 similar to vacuum source 70 of FIG. 5.

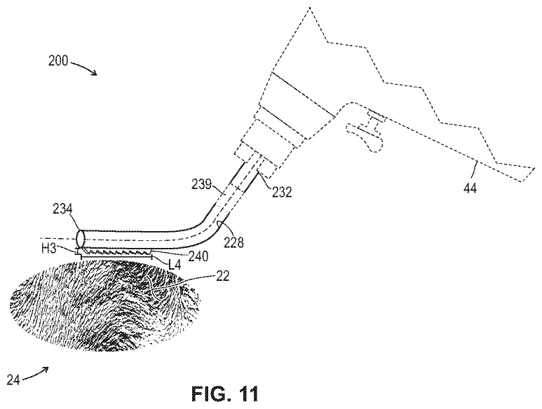

In some embodiments, a device 200 for decorticating bone is provided, as shown in FIG. 11. The device is similar to device 20 of FIGS. 1-8, however device 200 does not include a channel configured to receive bone material defined by the tubular member. Instead, the device is a stand alone rasping tool. The device includes a tubular member 228. The tubular member includes a proximal end 232, a distal end 234 and a longitudinal axis CC disposed therebetween. The proximal end is configured for engagement with oscillating tool 44, as described above with regard to FIGS. 1-8.

The device includes a rasp 240 that is similar to rasp 40 of FIGS. 1-8 that is configured for oscillating movement to decorticate bone at the bone site, as shown in FIG. 11. In some embodiments, the rasp is located on an exterior surface of the distal end of the tubular member. In some embodiments, the rasp is located on an exterior surface of the distal end of the tubular member and runs parallel with the distal end and perpendicular to the distal opening of the tubular member.

In some embodiments, the rasp can have a length L4. In some embodiments, the length L4 can be from about 2 mm to about 50 mm. In some embodiments, the length L4 can be from about 2 mm to about 40 mm, from about 2 mm to about 30 mm, from about 2 mm to about 20 mm, from about 2 mm to about 10 mm, from about 10 mm to about 50 mm, from about 10 mm to about 40 mm, from about 10 mm to about 30 mm, from about 10 mm to about 20 mm, from about 20 mm to about 50 mm, or from about 20 mm to about 40 mm. In some embodiments, the length L4 can be from about 2, 4, 6, 8, 10, 12, 14, 16, 18, 20, 22, 24, 26, 28, 30, 32, 34, 36, 38, 40, 42, 44, 46, 48 to about 50 mm.

In some embodiments, the rasp can have a certain height H3. In some embodiments, the height H3 can be from about 1 mm to about 8 mm, from about 1 mm to about 6 mm, or from about 1 mm to about 4 mm. In some embodiments, the height H3 can be from about 1, 2, 3, 4, 5, 6, 7 to about 8 mm.

In some embodiments, the rasp has various surface configurations and materials, and can include the surface configurations and materials disclosed above with regard to rasp 40 of FIGS. 1-8.

In some embodiments, the rasp can be monolithic with the distal end of the tubular member. For example, the rasp can be formed from the tubular member and can be made from the same material. In some embodiments, the rasp and the tubular member can be made via over-molding techniques or 3-D printing such that the rasp and the tubular member are formed from either the same or different material.

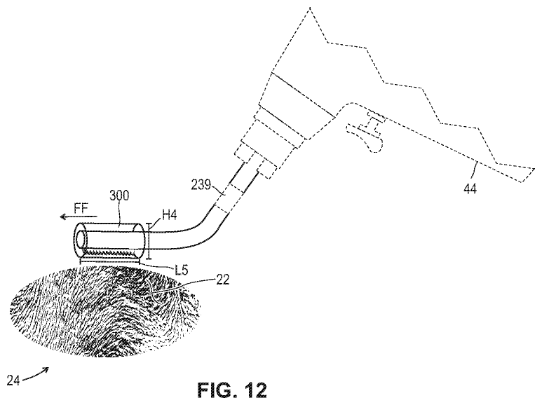

The tubular member can have a flexible portion 239, as shown in FIGS. 11 and 12, similar to flexible portion 39 of FIG. 4 that allows the rasp to be angled to enhance the rasping of bone by the rasp. In this way, the device can be positioned in confined area of the bone (e.g., the spine).

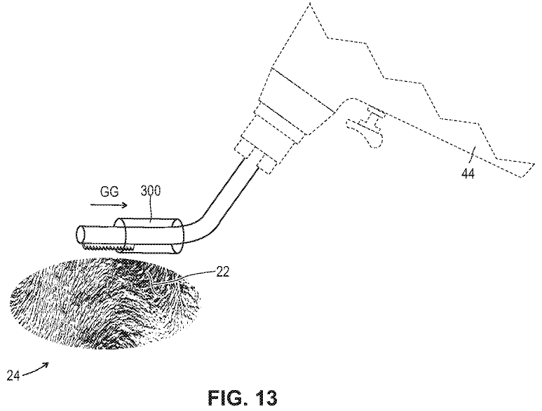

In some embodiments, devices 20 and/or 200 can include a sleeve 300, as shown in FIGS. 12 and 13. The sleeve is configured to slidably receive the tubular member and to enclose the rasp when the rasp is not in use, as shown in FIG. 12 and to retract in order to expose at least a portion of the rasp for application to the bone site, as shown in FIG. 13. In application, the sleeve is moved in a distal direction, as shown by arrow FF in FIG. 12 to enclose or partially enclose the rasp. When the practitioner is about to implement the rasp, the practitioner will then move the sleeve in a proximal direction, as shown by arrow GG in FIG. 13 to expose at least all or a portion of the rasp for use. In some embodiments, the sleeve can have a locking feature to fix the sleeve in a certain position for use.

In some embodiments, the sleeve can have a length L5, as shown in FIG. 12. In some embodiments, the sleeve length is the same, less than or greater than the length of the rasp. In some embodiments, length L5 is from about 2 mm to about 60 mm. In some embodiments, the length L5 can be from about 2, 4, 6, 8, 10, 12, 14, 16, 18, 20, 22, 24, 26, 28, 30, 32, 34, 36, 38, 40, 42, 44, 46, 48, 50, 52, 54, 56, 58 to about 60 mm.

In some embodiments, the sleeve can have a certain height H4, as shown in FIG. 12. In some embodiments, the height H4 is greater than the combined height of the rasp and the diameter of the tubular member. In some embodiments, the height H4 can be from about 6 mm to about 40 mm. In some embodiments, the height H4 can be from about 6, 8, 10, 12, 14, 16, 18, 20, 22, 24, 26, 28, 30, 32, 34, 36, 38 to about 40 mm.

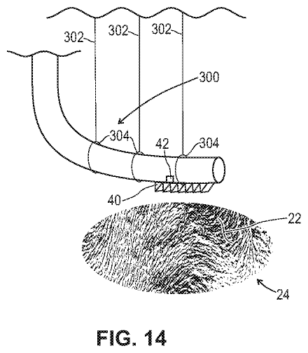

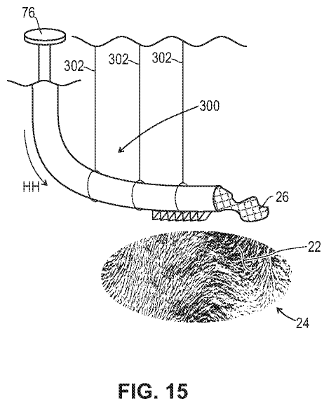

In some embodiments, a device 300 for decorticating bone is provided, as shown in FIGS. 14-15. The device is similar to device 20 of FIGS. 1-8 and includes all of the features of FIGS. 1-8 in addition to at least one guidewire 302. In some embodiments, the at least one guidewire is configured to be inserted percutaneously at the bone site which can be a spine of a patient. In some embodiments, the at least one guidewire can include 1, 2, 3, 4, 5, 6, 7, 8, 9 or 10 guidewires. The at least one guidewire includes a loop 304 at a distal end to allow the tubular member to be slidably received in the loop. Thus, the tubular member may be concentric with the loop as shown in FIGS. 14 and 15. The loop can, in some embodiments, be adjacent to the tubular member. In some embodiments, the rasp can be on the exterior surface of at least a portion of the tubular member and the rasp can decorticate the bone.

In some embodiments, the device can be manufactured with the pressure sensor, as shown in FIG. 14 or without the pressure sensor, as shown in FIG. 15. In use, a dilator can be used to create an opening above the surgical site percutaneously. The at least one guidewire is then inserted and positioned at the bone site. The device is then slidably received in the loop of the at least one guidewire. The device is then oscillated by the oscillating tool such that the rasp decorticates the bone site. The suction tool can be used to remove decorticated bone particulate and then the suction tool can be removed from the channel of the tubular member of the device. The plunger is then inserted and is moved in a distal direction, as shown by arrow HH in FIG. 15 to dispense the bone material at the bone site. In some embodiments, screws and/or rods can be inserted into the bone site. In some embodiments, screws can be inserted into the bone site and the incision site can be reused for placement of a rod or rods. In some embodiments, the device is guided by the at least one guidewire at a posterolateral bone site.

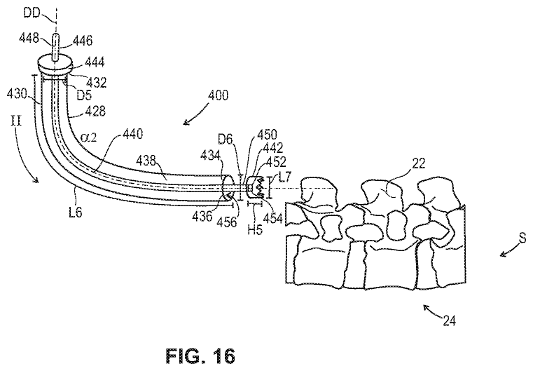

In some embodiments, a device 400 for decortication of a bone site is provided, as shown in FIG. 16, similar to device 20 of FIGS. 1-8. In some embodiments, the device can be inserted percutaneously and can be navigated parallel to the spine S, as shown in FIG. 16. The device comprises an elongated tubular member 428 similar to tubular member 28 of FIGS. 1-8. In some embodiments, the tubular member is flexible. In some embodiments, the tubular member can be a curved cannula.

The tubular member comprises a proximal end 430 defining a proximal opening 432, a distal end 434 defining a distal opening 436, a channel 438 and a longitudinal axis DD disposed therebetween. The channel is configured to receive a wire member 440 having a rasp 442 configured for movement for decortication of the bone site, as described herein. The proximal opening is configured for engagement with a handle 444 having a centrally located opening 446 for engagement with the wire member.

The proximal opening has a diameter D5 and the distal opening has a diameter D6. In some embodiments, diameters D5 and D6 can be the same size, D5 can be greater than D6 or D6 can be greater than D5. In some embodiments, D5 and D6 can be from about 4 mm to about 30 mm, from about 4 mm to about 20 mm, from about 4 mm to about 10 mm, from about 10 mm to about 30 mm, from about 10 mm to about 20 mm, from about 10 to about 15 mm, from about 15 mm to about 30 mm, from about 15 to about 25 mm, from about 15 mm to about 20 mm, from about 20 mm to about 30 mm or from about 20 mm to about 25 mm. In some embodiments, diameters D5 and D6 can be from about 4, 5, 6, 7, 8, 9, 10, 11, 12, 13, 14, 15, 16, 17, 18, 19, 20, 21, 22, 23, 24, 25, 26, 27, 28, 29 to about 30 mm.

In some embodiments, the tubular member can have a length L6. The length L6 can be from about 1 inch to about 20 inches, from about 1 to about 15 inches, from about 1 to about 10 inches, from about 1 to about 5 inches, from about 5 to about 20 inches, from about 5 to about 15 inches, from about 5 to about 10 inches, from about 10 to about 20 inches, from about 10 to about 15 inches, or from about 15 to about 20 inches. In some embodiments, the length L6 can be from about 1, 2, 3, 4, 5, 6, 7, 8, 9, 10, 11, 12, 13, 14, 15, 16, 17, 18, 19 to about 20 inches.

In some embodiments, the tubular member can be angled and have an angle α2 from about 10 to about 60 degrees. In some embodiments, α1 can be from about 10, 12, 14, 16, 18, 20, 22, 24, 26, 28, 30, 32, 34, 36, 38, 40, 42, 44, 46, 48, 50, 52, 54, 56, 58 to about 60 degrees.

The wire member includes a proximal end 448 and a distal end 450. The distal end includes the rasp. In some embodiments, the wire member can be the same, greater than, or less than in length as the tubular member. In some embodiments, the wire member can be an electrical wire that sends signals to a processor, as described herein.

The rasp is configured for movement for decortication of the bone site. The rasp is similar to rasp 40 of FIGS. 1-8. In some embodiments, the rasp can have a length L7. In some embodiments, the length L7 can be from about 2 mm to about 20 mm. In some embodiments, the length L7 can be from about 2, 4, 6, 8, 10, 12, 14, 16, 18 to about 20 mm.

In some embodiments, the rasp can have a certain height H5. In some embodiments, the height H5 can be from about 1 mm to about 8 mm, from about 1 mm to about 6 mm, or from about 1 mm to about 4 mm. In some embodiments, the height H5 can be from about 1, 2, 3, 4, 5, 6, 7 to about 8 mm.

In some embodiments, the rasp has various surface configurations and materials, and can include the surface configurations and materials disclosed above with regard to rasp 40 of FIGS. 1-8.

In some embodiments, the rasp can be monolithic with the distal end of the wire member. For example, the rasp can be formed from the wire member and can be made from the same material. In some embodiments, the rasp and the wire member can be made via over-molding techniques or 3-D printing such that the rasp and the wire member are formed from either the same or different material. In some embodiments, the device comprises a locking mechanism to lock the wire member and the rasp at a specific location within or outside of the tubular member.

The rasp comprises a pressure sensor 452 that includes a transmitter 454. The pressure sensor is configured to at least measure pressure from the movement of the wire member when a predetermined pressure or a predetermined pressure change is measured at the bone site. The pressure sensor and the transmitter are the same as pressure sensor 42 and transmitter 48 described above with regard to FIGS. 1-8. In some embodiments, the pressure sensor is configured to transmit electronic signals to a processor of a computer, as described above with regard to FIGS. 1-8.

In some embodiments, the rasp and/or the device can include 1, 2, 3, 4, 5, 6, 7, 8, 9 or 10 sensors. In some embodiments, the rasp and/or the device can include additional sensors such as, for example, an image sensor, a motion sensor, a temperature sensor, and/or a humidity sensor. In some embodiments, the pressure sensor is coupled to an oscillating tool 44, as described herein.

In some embodiments, device includes a slot rail 456 configured to self-guide the wire member and the rasp. In some embodiments, the slot rail enables the wire member to travel through the channel of the tubular member without shaking, bending or other factors that interfere with the movement of the wire member.

In some embodiments, the tubular members described above can be folding cannulas. The folding cannula can be made of a memory shape polymer and/or alloy to allow the folding cannula to move from an unfolded configuration to a folded configuration without the need for a locking mechanism. Memory shape polymers include, but are not limited to polyethers, polyacrylates, polyamides, polysiloxanes, polyurethanes, polyethers amides, polyurethane/ureas, polyether esters, polynorborene, cross-linked polymers such as cross-linked polyethylene and cross-linked poly(cyclooctene), inorganic-organic hybrid polymers, and copolymers such as urethane/butadiene copolymers, styrene-butadiene copolymers. Memory shape alloys include, but are not limited to TiNi, CuZnAI, and FeNiAI alloys. In some embodiments, the folding cannula can be fabricated by injection molding of plastic materials comprising rigid, surgical grade plastic and/or metal materials.

In some embodiments, components of the device may be made from materials, such as for example, polyurethane, polyurea, polyether(amide), PEBA, thermoplastic elastomeric olefin, copolyester, and styrenic thermoplastic elastomer, steel, aluminum, stainless steel, titanium, nitinol, metal alloys with high non-ferrous metal content and a low relative proportion of iron, carbon fiber, glass fiber, plastics, ceramics or combinations thereof. The folding cannula, funnel portion, plunger or tubular member may optionally include one or more tapered regions. In various embodiments, these components may be blunt, beveled, diamond point, ball tip, trocar tip, etc. These components may also have a tip style vital for accurate treatment of the patient depending on the surgical site. Examples of tip styles include, for example, Trephine, Coumand, Veress, Huber, Seldinger, Chiba, Francine, Bias, Crawford, deflected tips, Hustead, Lancet, or Tuohey. In some embodiments, the bone material dispensing device and tray can be made from materials that allow the bone material dispensing device to be reusable, or alternatively made from materials that allow for a single, disposable use.

In some embodiments, the shape of the folding cannula may be selected for particular applications. Such shape and configuration may include, for example, the basic shape of a folding cannula (e.g., a tubular shaped cannula).

### Kit

In various embodiments, a kit can be provided containing the device prefilled with bone material or the kit can contain the device. In some embodiments, the kit may include additional parts along with the device such as the bone material (e.g., bone graft) and dilators (e.g., wipes, needles, etc.). The kit may include the device in a first compartment. The second compartment may include the bone material sealed in a container, along with a vial containing diluent and any other delivery instruments needed for the localized delivery. A third compartment may include gloves, drapes, wound dressings and other procedural supplies for maintaining sterility of the implanting process, as well as an instruction booklet, which may include a chart that shows how to implant the bone material. A fourth compartment may include additional needles, fasteners, and/or sutures. Each tool may be separately packaged in a plastic pouch that is radiation sterilized. A fifth compartment may include an agent for radiographic imaging. A cover of the kit may include illustrations of the implanting procedure and a clear plastic cover may be placed over the compartments to maintain sterility.

### Methods

A method of decorticating a bone site is provided. The method can be employed with various delivery instruments and in a surgical treatment with a patient in a prone or supine position, and/or employ various surgical approaches to the spine, including anterior, posterior, posterior mid-line, direct lateral, and/or antero-lateral approaches, and in other body regions. The method may also be employed with procedures for treating the lumbar, cervical, thoracic, sacral and pelvic regions of a spinal column. The method may also be used on animals, bone models and other non-living substrates, such as, for example, in training, testing and demonstration.

The method comprises inserting at the bone site a device for decorticating bone at the bone site, the device comprising an elongated tubular member, the tubular member comprising a rasp configured for oscillating movement of the rasp for decortication of the bone site, and the rasp comprising a pressure sensor configured to at least measure pressure from the oscillating movement of the rasp when a predetermined pressure or a predetermined pressure change is measured at the bone site.

In some embodiments, the method further comprise oscillating the tubular member to decorticate the bone site. In some embodiments, the method further comprises applying suction to the tubular member to remove decorticated bone particles. In some embodiments, the method further comprises retracting a sleeve partially enclosing the rasp to expose the rasp for decorticating bone. In some embodiments, the method further comprises extending a flexible plunger through a channel of the tubular member to dispense a bone material to the bone site.

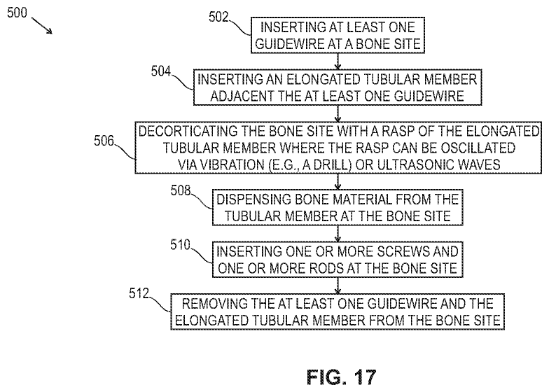

In some embodiments, as shown in the block diagram of FIG. 17, a method of decorticating a bone site is provided 500. At least one guidewire is inserted at a bone site 502. An elongated tubular member is then inserted adjacent the at least one guidewire 504. Next, the bone site is decorticated with a rasp of the elongated tubular member where the rasp can be oscillated via vibration or ultrasound waves 506. Bone material is then dispensed from the elongated tubular member at the bone site 508. In some embodiments, the bone material can be dispensed via a flexible plunger. One or more screws are inserted and/or one or more rods are inserted at the bone site 510. The at least one guidewire is then removed and the elongated tubular member is removed from the bone site 512.

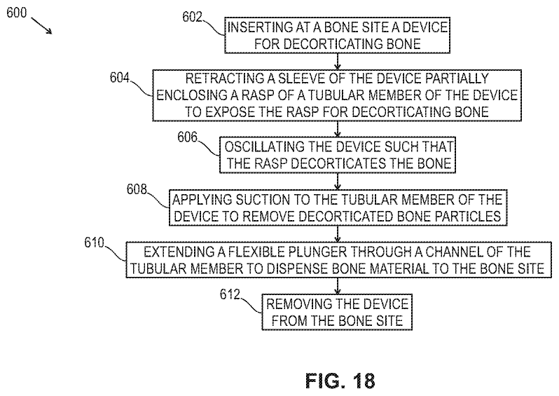

In some embodiments, as shown in the block diagram of FIG. 18, a method of decorticating bone 600 is provided. The device for decorticating bone is inserted at a bone site 602. A sleeve of the device is retracted that partially encloses a rasp of a tubular member of the device to expose the rasp for decorticating bone 604. The device is oscillated such that the rasp decorticates the bone site 606. Suction is then applied via a suction tool disposed within a channel of the tubular member to remove decorticated bone particles 608. A flexible plunger is then extended through the tubular member to dispense bone material out of the tubular member and into the bone site 610. The device is then removed from the bone site 612.

The bone material may be used in a minimally invasive procedure via placement through a small incision, via delivery through the dilators, or other means. The size and shape may be designed with restrictions on delivery conditions. For example, the bone material may be percutaneously delivered to the surgical site, and in some cases, the surgical site is the posterior spine.

In some embodiments, the bone material may be used in healing vertebral compression fractures, interbody fusion, minimally invasive procedures, posterolateral fusion, correction of adult or pediatric scoliosis, treating long bone defects, osteochondral defects, ridge augmentation (dental/craniomaxillofacial, e.g. edentulous patients), beneath trauma plates, tibial plateau defects, filling bone cysts, wound healing, around trauma, contouring (cosmetic/plastic/reconstructive surgery), and others.

Generally, the bone material may be applied to a pre-existing defect, to a created channel, or to a modified defect. Thus, for example, a channel may be formed in a bone, or a pre-existing defect may be cut to form a channel, for receipt of the bone material. The bone material may be configured to match the channel or defect. In some embodiments, the configuration of bone material may be chosen to match the channel. In other embodiments, the channel may be created, or the defect expanded or altered, to reflect a configuration of the bone material. The bone material may be placed in the defect or channel and, optionally, coupled using attachment mechanisms.

The bone material can be mixed with liquid material and optionally a therapeutic agent until a desired consistency of the bone material is achieved (e.g., putty, paste, etc.). The bone material can be mixed with a suitable diluent and then loaded. The cannula may have enough space to allow for the bone material and a volume of diluent to be mixed. In some embodiments, the diluent includes dextrose, other sugars including but not limited to sucrose, fructose, glucose, lactated ringer's, polyols including, but not limited to, mannitol, xylitol, sorbitol, maltitol, lactitol, polysaccharides including, but not limited to, native or pre-gelatinized starch, maltodextrins, cyclodextrins, mineral compounds including, but not limited to, dicalcium or tricalcium phosphate, either dihydrate or anhydrous, cellulose derivatives including, but not limited to, microcrystalline cellulose, lactoses either monohydrates thereof or anhydrous, as well as their mixtures such as dicalcium phosphate dihydrate, mannitol, pre-gelatinized maize starch, microcrystalline cellulose and their mixtures, water and/or NaCl (saline). In some embodiments, the saline is 0.90% saline or 0.45% saline. In some embodiments, other delivery vehicles can be used for example, D5W (dextrose in 5% water), D5NS (dextrose in 5% water and normal saline) and D5W/½NS (D5W and ½ normal saline), blood, mesenchymal stem cells, or the like.

In various embodiments, one or more components of the device are sterilized by radiation in a terminal sterilization step in the final packaging. Terminal sterilization of a product provides greater assurance of sterility than from processes such as an aseptic process, which requires individual product components to be sterilized separately and the final package assembled in a sterile environment.

In various embodiments, gamma radiation is used in the terminal sterilization step, which involves utilizing ionizing energy from gamma rays that penetrate deeply into the bone material dispensing device. Gamma rays are highly effective in killing microorganisms, they leave no residues, nor do they have sufficient energy to impart radioactivity to the apparatus. Gamma rays can be employed when the device is in the package and gamma sterilization does not require high pressures or vacuum conditions, thus, package seals and other components are not stressed. In addition, gamma radiation eliminates the need for permeable packaging materials.

In various embodiments, electron beam (e-beam) radiation may be used to sterilize one or more components of the bone material dispensing device. E-beam radiation comprises a form of ionizing energy, which is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly-charged streams of electrons generated by the acceleration and conversion of electricity.

Other methods may also be used to sterilize the device including, but not limited to, gas sterilization such as, for example, with ethylene oxide or steam sterilization.

The device can be used to treat a variety of conditions including osteoporosis, bone fracture repair or healing, dental procedures for which increased bone formation in the jaw is of clinical benefit, repair of craniofacial bone defects induced by trauma or congenital defects such as cleft palate/lip, and a number of other musculoskeletal disorders where native bone growth is inadequate, which will be evident to those of ordinary skill in the art. The bone material can be administered to treat open fractures and fractures at high risk of non-union, and in subjects with spinal disorders, including subjects in need of spine fusion (e.g., anterior lumbar interbody fusion, posterior lumbar spinal fusion, and cervical spine fusion) or subjects having degenerative disc disease or arthritis affecting the lumbar and cervical spine.

### Bone Material

In some embodiments, the bone material can be demineralized bone material. The demineralized bone material can comprise demineralized bone, powder, chips, granules, shards, fibers or other shapes having irregular or random geometries. These can include, for example, substantially demineralized, partially demineralized, or fully demineralized cortical and cancellous bone. These also include surface demineralization, where the surface of the bone construct is substantially demineralized, partially demineralized, or fully demineralized, yet the body of the bone construct is fully mineralized. The configuration of the bone material can be obtained by milling, shaving, cutting or machining whole bone as described in, for example, U.S. Pat. No. 5,899,939. The entire disclosure is herein incorporated by reference into the present disclosure.

In some embodiments, the bone material can comprise elongated demineralized bone fibers having an average length to average thickness ratio or aspect ratio of the fibers from about 50:1 to about 1000:1. In overall appearance the elongated demineralized bone fibers can be round, spherical, granular, elongated, powders, chips, fibers, cylinders, threads, narrow strips, thin sheets, or a combination thereof. In some embodiments, the bone material comprises elongated demineralized bone fibers and chips. In some embodiments, the bone material comprises fully demineralized fibers and surface demineralized chips. In some embodiments, the ratio of fibers to chips or powders is from about 5, 10, 15, 20, 25, 30, 35, 40, or 45 fibers to about 30, 35, 40, 45, 50, 55, 60, 65, or 70 chips.

In some embodiments, the bone material comprises demineralized bone matrix fibers and demineralized bone matrix chips in a 30:60 ratio. In some embodiments, the bone material comprises demineralized bone matrix fibers and demineralized bone matrix chips in a ratio of 25:75 to about 75:25 fibers to chips.

In some embodiments, the bone material can be an inorganic material, such as an inorganic ceramic and/or bone substitute material. Exemplary inorganic materials or bone substitute materials include but are not limited to aragonite, dahlite, calcite, brushite, amorphous calcium carbonate, vaterite, weddellite, whewellite, struvite, urate, ferrihydrate, francolite, monohydrocalcite, magnetite, goethite, dentin, calcium carbonate, calcium sulfate, calcium phosphosilicate, sodium phosphate, calcium aluminate, calcium phosphate, hydroxyapatite, alpha-tricalcium phosphate, dicalcium phosphate, β-tricalcium phosphate, tetracalcium phosphate, amorphous calcium phosphate, octacalcium phosphate, BIOGLASS™ fluoroapatite, chlorapatite, magnesium-substituted tricalcium phosphate, carbonate hydroxyapatite, substituted forms of hydroxyapatite (e.g., hydroxyapatite derived from bone may be substituted with other ions such as fluoride, chloride, magnesium sodium, potassium, etc.), or combinations or derivatives thereof.

In some embodiments, the bone material can comprise mineral particles, which comprise tricalcium phosphate and hydroxyapatite in a ratio of about 80:20 to about 90:10. In some embodiments, the mineral particles can comprise tricalcium phosphate and hydroxyapatite in a ratio of about 70:30 to about 95:5. In some embodiments, the mineral particles can comprise tricalcium phosphate and hydroxyapatite in a ratio of about 85:15.

In some embodiments, the bone material may be seeded with harvested bone cells and/or bone tissue, such as for example, cortical bone, autogenous bone, allogenic bones and/or xenogeneic bone while it is mixed.

In some embodiments, the bone material may be mixed with one or more therapeutic agents, for example, an anti-inflammatory agent, an analgesic agent, an osteoinductive growth factor, an antimicrobial agent or a combination thereof. Osteoinductive agents include one or more members of the family of Bone Morphogenetic Proteins (“BMPs”). BMPs are a class of proteins thought to have osteoinductive or growth-promoting activities on endogenous bone tissue, or function as pro-collagen precursors. Known members of the BMP family include, but are not limited to, BMP-1, BMP-2, BMP-3, BMP-4, BMP-5, BMP-6, BMP-7, BMP-8, BMP-9, BMP-10, BMP-11, BMP-12, BMP-13, BMP-14 (GDF-5), BMP-15, BMP-16, BMP-17, BMP-18 as well as polynucleotides or polypeptides thereof, as well as mature polypeptides or polynucleotides encoding the same.

BMPs utilized as osteoinductive agents comprise one or more of BMP-1; BMP-2; BMP-3; BMP-4; BMP-5; BMP-6; BMP-7; BMP-8; BMP-9; BMP-10; BMP-11; BMP-12; BMP-13; BMP-15; BMP-16; BMP-17; or BMP-18; as well as any combination of one or more of these BMPs, including full length BMPs or fragments thereof, or combinations thereof, either as polypeptides or polynucleotides encoding the polypeptide fragments of all of the recited BMPs. The isolated BMP osteoinductive agents may be administered as polynucleotides, polypeptides, full length protein or combinations thereof.

Indeed, the osteoinductive factors are the recombinant human bone morphogenetic proteins (rhBMPs) because they are available in unlimited supply and do not transmit infectious diseases. In some embodiments, the bone morphogenetic protein is a rhBMP-2, rhBMP-4, rhBMP-7, or heterodimers thereof. Recombinant BMP-2 can be used at a concentration of about 0.4 mg/mL to about 10.0 mg/mL, preferably about 1.5 mg/mL.

The bone material may include or be mixed with one or more members from the TGF-β superfamily. For example, the matrix may include AMH, ARTN, GDF1, GDF10, GDF11, GDF15, GDF2, GDF3, GDF3A, GDF5, GDF6, GDF7, GDF8, GDF9, GDNF, INHA, INHBA, INHBB, INHBC, INHBE, LEFTY1, LEFTY2, MSTN, NODAL, NRTN, PSPN, TGFB1, TGFB2, TGFB3, FGF, basic FGF, VEGF, insulin-like growth factor, EGF, PDGF, nerve growth factor or combinations thereof.

The bone material may include or be mixed with a therapeutic agent including, but not limited to, IL-1 inhibitors, such Kineret® (anakinra), which is a recombinant, non-glycosylated form of the human interleukin-1 receptor antagonist (IL-1Ra), or AMG 108, which is a monoclonal antibody that blocks the action of IL-1. The bone material may include or be mixed with therapeutic agents including excitatory amino acids such as glutamate and aspartate, antagonists or inhibitors of glutamate binding to NMDA receptors, AMPA receptors, and/or kainate receptors. The bone material may include or be mixed with therapeutic agents to reduce inflammation including but not limited to interleukin-1 receptor antagonists, thalidomide (a TNF-α release inhibitor), thalidomide analogues (which reduce TNF-α production by macrophages), quinapril (an inhibitor of angiotensin II, which upregulates TNF-α), interferons such as IL-11 (which modulate TNF-α receptor expression), or aurin-tricarboxylic acid (which inhibits TNF-α).

The bone material may include or be mixed with a therapeutic agent including, but not limited to, an analgesic agent. Examples of analgesic agents include, but are not limited to, acetaminophen, tramadol, lidocaine, bupivacaine, ropivacaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, levorphanol, meperidine, methadone, morphine, nalbuphine, opium, oxycodone, papaveretum, pentazocine, pethidine, phenoperidine, piritramide, dextropropoxyphene, remifentanil, sufentanil, tilidine, tramadol, codeine, dihydrocodeine, meptazinol, dezocine, eptazocine, flupirtine or a combination thereof.

The bone material may include or be mixed with a therapeutic agent including, but not limited to, an anti-inflammatory agent. An example of an anti-inflammatory agent includes, but is not limited to, clonidine, sulindac, sulfasalazine, naroxyn, diclofenac, indomethacin, ibuprofen, flurbiprofen, ketoprofen, aclofenac, aloxiprin, aproxen, aspirin, diflunisal, fenoprofen, mefenamic acid, naproxen, phenylbutazone, piroxicam, meloxicam, salicylamide, salicylic acid, desoxysulindac, tenoxicam, ketoralac, clonidine, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, triflumidate, fenamates (mefenamic acid, meclofenamic acid), nabumetone, celecoxib, etodolac, nimesulide, apazone, gold, tepoxalin; dithiocarbamate, or a combination thereof.

Anti-inflammatory agents also include steroids, such as for example, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, dexamethasone 21-acetate, dexamethasone 21-phosphate di-Na salt, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide or a combination thereof.

The bone material may include or be mixed with a therapeutic agent including, but not limited to, a statin. Examples of a useful statin include, but are not limited to, atorvastatin, simvastatin, pravastatin, cerivastatin, mevastatin (see U.S. Pat. No. 3,883,140, the entire disclosure is herein incorporated by reference), velostatin (also called synvinolin; see U.S. Pat. Nos. 4,448,784 and 4,450,171 these entire disclosures are herein incorporated by reference), fluvastatin, lovastatin, rosuvastatin and fluindostatin (Sandoz XU-62-320), dalvastain (EP Application Publication No. 738510 A2, the entire disclosure is herein incorporated by reference), eptastatin, pitavastatin, or pharmaceutically acceptable salts thereof or a combination thereof. In various embodiments, the statin may comprise mixtures of (+)R and (−)-S enantiomers of the statin. In various embodiments, the statin may comprise a 1:1 racemic mixture of the statin.

In some embodiments, the bone material can include an antimicrobial agent. In some embodiments, the antimicrobial agent can include one or more of triclosan, also known as 2,4,4′-trichloro-2′-hydroxydiphenyl ether, chlorhexidine and its salts, including chlorhexidine acetate, chlorhexidine gluconate, chlorhexidine hydrochloride, and chlorhexidine sulfate, silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine, polymyxin, tetracycline, aminoglycosides, such as tobramycin and gentamicin, rifampicin, bacitracin, neomycin, chloramphenicol, miconazole, quinolones such as oxolinic acid, norfloxacin, nalidixic acid, pefloxacin, enoxacin and ciprofloxacin, penicillins such as oxacillin and pipracil, nonoxynol 9, fusidic acid, cephalosporins, or combinations thereof.

Examples of antimicrobial agents include, by way of illustration and not limited to, acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefacior, cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; dindamycin palmitate hydrochloride; clindamycin phosphate; clofazimine; cloxacillin benzathine; cloxacillin sodium; chlorhexidine, cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracydine calcium; oxytetracycline hydrochloride; paldimycin; parachlorophenol; paulomycin; pefloxacin; pefloxacin mesylate; penamecillin; penicillins such as penicillin G benzathine, penicillin G potassium, penicillin G procaine, penicillin G sodium, penicillin V, penicillin V benzathine, penicillin V hydrabamine, and penicillin V potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracydine nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; zorbamycin; or combinations thereof.

The antimicrobial agent in the bone material can be an antiviral agent that can be mixed with the bone material. Antiviral agents can include, but are not limited to, vidarabine, acyclovir, famciclovir, valacyclovir, gancyclovir, valganciclovir, nucleoside-analog reverse transcriptase inhibitors (such as AZT (zidovudine), ddI (didanosine), ddC (zalcitabine), d4T (stavudine), and 3TC (lamivudine)), nevirapine, delavirdine, protease inhibitors (such as, saquinavir, ritonavir, indinavir, and nelfinavir), ribavirin, amantadine, rimantadine, neuraminidase inhibitors (such as zanamivir and oseltamivir), pleconaril, cidofovir, foscamet, and/or interferons.

It is to be understood that persons skilled in the art will recognize that two or more embodiments may be combined without departing from the spirit and scope of the disclosure.

Although the invention has been described with reference to embodiments, persons skilled in the art will recognize that changes may be made in form and detail without departing from the spirit and scope of the disclosure.

What is claimed is:

1. A device for decortication of a bone site, the device comprising an elongated tubular member, the tubular member comprising a rasp configured for oscillating movement of the rasp for decortication of the bone site, and the rasp comprising a pressure sensor configured to at least measure pressure from the oscillating movement of the rasp when a predetermined pressure or a predetermined pressure change is measured at the bone site.

2. The device of claim 1, wherein the pressure sensor is coupled to an oscillating tool, the oscillating tool configured to engage the elongated tubular member and reduce or discontinue oscillation of the rasp when the predetermined pressure or the predetermined pressure change is measured at the bone site.

3. The device of claim 1, wherein the rasp is disposed at an exterior surface of the elongated tubular member.

4. The device of claim 1, wherein the device further comprises a suction tool configured to engage a distal end of the elongated tubular member to remove decorticated bone particles from the bone site.

5. The device of claim 2, wherein the oscillating tool comprises an ultrasonic energy source configured to supply the rasp with an ultrasonic energy, the ultrasonic energy source coupled to a transducer to transfer the ultrasonic energy from the ultrasonic energy source to the rasp of the tubular member.

6. The device of claim 1, wherein the elongated tubular member engages a guide wire.

7. The device of claim 6, wherein the guidewire is configured to be inserted percutaneously to the bone site which comprises a spine of a patient.

8. The device of claim 1, wherein the tubular member is flexible and comprises a channel configured to receive a plunger for dispensing a bone material loaded within the channel.

9. The device of claim 8, wherein the channel is configured to engage a suction tool to remove decorticated bone particles from the bone site.

10. The device of claim 1, further comprising a flexible plunger wherein at least a portion of the plunger is configured to slide within the tubular member, and the flexible plunger is configured to dispense a bone material from a distal end of the tubular member at the bone site.

11. The device of claim 1, wherein the device further comprises a sleeve enclosing the rasp, the sleeve configured to retract to expose at least a portion of the rasp.

12. The device of claim 1, wherein the device further comprises a sleeve configured to receive the tubular member.

13. The device of claim 1, wherein the rasp is configured to oscillate (i) axially relative to a longitudinal axis of the tubular member; (ii) radially relative to the longitudinal axis of the tubular member; or (iii) diagonally relative to the longitudinal axis of the tubular member.

14. The device of claim 1, wherein the rasp is configured to vibrate causing the rasp to move in multiple directions.

15. A device for decortication of a bone site, the device comprising an elongated tubular member, the tubular member having a channel configured to receive a wire member having a rasp configured for movement for decortication of the bone site, and the rasp comprising a pressure sensor configured to at least measure pressure from the movement of the wire member when a predetermined pressure or a predetermined pressure change is measured at the bone site.

16. A method for decorticating a bone site, the method comprising inserting at the bone site a device for decorticating bone at the bone site, the device comprising an elongated tubular member, the tubular member comprising a rasp configured for oscillating movement of the rasp for decortication of the bone site, and the rasp comprising a pressure sensor configured to at least measure pressure from the oscillating movement of the rasp when a predetermined pressure or a predetermined pressure change is measured at the bone site.

17. The method of claim 16, further comprising oscillating the tubular member to decorticate the bone site.

18. The method of claim 16, further comprising applying suction to the tubular member to remove decorticated bone particles.

19. The method of claim 16, further comprising retracting a sleeve partially enclosing the rasp to expose the rasp for decorticating bone.

20. The method of claim 16, further comprising extending a flexible plunger through a channel of the tubular member to dispense a bone material to the bone site.
